(19) United States
(12) Patent Application Publication
(10) Pub. No.: US 2005/0137581 A1
(43) Pub. Date: Jun. 23, 2005

(54) Liposuction/tubing coupling for providing rotational movement

(76) Inventor: Kouros Azar, Thousand Oaks, CA (US)

Correspondence Address: MATTHEW A. NEWBOLES;STETINA BRUNDA GARRED & BRUCKER, Suite 250, 75 Enterprise, Aliso Viejo, CA, 92656, US

(21) Appl. No.: 10/738,801

(22) Filed: Dec. 17, 2003

(51) Int. Cl. A61M 25/00; A61M 1/00

(52) U.S. Cl. 604/542; 604/523

(57) ABSTRACT

A liposuction cannula/suction tubing coupling is disclosed that enables the tubing to rotate freely about the juncture at which the tubing is coupled to the cannula. Such coupling may be formed as a separate fitting that is interconnected intermediate the liposuction cannula and the suction tubing, or integrally formed as part of the liposuction cannula, on one hand, or on the suction tubing, on the other hand.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND OF THE INVENTION

Surgical procedures involving lipoplasty are well-known in the art. Perhaps the most well-known and extensively performed of such procedures is tumescent liposuction, which employs a large volume of fluid, generally comprised of a very dilute epinephrine solution and a dilute anesthetic solution, that is infiltrated into a target fat compartment to produce swelling and firmness therein. In this regard, such fluid is operative to maintain the fat in a tumescent state while at the same time producing vasoconstriction and localized anesthesia. While the fat is maintained in such tumescent state, a liposuction cannula is systematically inserted into the fat compartment in a series of elongate paths. Such cannula, which is connected to a suction device, namely, surgical tubing operatively connected with a vacuum or suction mechanism, extracts the suspended fatty tissue in elongate cylindrical portion that eventually creates a network of many small tunnels running throughout the targeted fat compartment. Over time, within the targeted fat compartment, the series of tunnels formed collapse and ultimately cause the compartment to assume a desired contour.

Advantageously, the tumescent liposuction technique allows for large amounts of fat to be removed from the body with virtually no blood loss. Additionally, the tumescent technique has further proven to be less painful, minimizes post-operative recovery time, and can produce optimal cosmetic results as compared with other lipoplasty procedures.

Despite the popularity of such procedure, tumescent liposuction does have the drawback of being labor intensive for the surgeon performing the operation. In this regard, the surgeon performing such procedure must continuously shove the elongate liposuction cannula in and out of a fatty tissue compartment in a repetitious manner while continuously moving the cannula radially about the compartment. To perform such procedure typically requires the surgeon to continuously move and manipulate the cannula, as well as the suction tubing coupled therewith. As a consequence, a continuous twisting and kinking of the suction tubing coupled to the liposuction cannula typically occurs during such procedure that must continuously be untwisted as the cannula and tubing are moved about a patient. Otherwise the liposuction procedure will not be properly performed. While advances have been made with respect to automated liposuction cannula deployment, such as power-assisted cannula produced by MicroAire Surgical Instruments, LLC, of Charlottesville, Va., the same still do not address the issue of the continuous twisting and kinking of the suction tubing that is rigidly connected to the liposuction cannula.

Accordingly, there is a substantial need in the art for a fitting or coupling operative to substantially eliminate the continuous twisting and kinking of suction tubing utilized in combination with a liposuction cannula. There is additionally a need in the art for such a device that is of exceedingly simple construction, easy to utilize, inexpensive to manufacture, and can be readily integrated with existing liposuction cannulas and suction tubing utilized therewith.

### BRIEF SUMMARY OF THE INVENTION

The present invention specifically addresses and alleviates the above-identified deficiencies in the art. In this regard, the present invention is directed to a fitting or coupling interposeable between a liposuction cannula and suction tubing coupled therewith that is operative to provide independent rotational movement of either the liposuction cannula and/or suction tubing about a common axis while maintaining such elements in an operatively conjoined configuration.

According to a preferred embodiment, the device will simply comprise a conventional liposuction cannula having a proximal end defining a fitting to which may be connected the distal-most end of a section of suction tubing. With respect to the latter, the same will be configured to interconnect with the proximal end of the liposuction cannula such that both the proximal end of the cannula and the tubing connected therewith are free to independently rotate, preferably about a common axis. Such interconnection defined by the fitting or coupling may comprise a complementary annular groove and collar formed respectively upon either the proximal end of the liposuction cannula or suction tubing. To facilitate such rotational movement, it is contemplated that a simple ball bearing structure may be utilized. In alternative configurations, the invention may comprise an interface integrally formed upon the proximal end of the cannula that may be coupled to conventional suction tubing and enable the cannula and tubing connected therewith to independently rotate, preferably about a common axis while remaining operatively interconnected. Alternatively, the distal most end of the suction tubing may be provided with such an interface integrally formed thereon for operative interconnection with a conventional liposuction cannula that likewise permits such independent rotational movement. In an additional embodiment of the present invention, the fitting or coupling may be a completely separate element operatively connectable between the cannula and tubing. Still further, the invention may comprise an embodiment where the proximal end of the liposuction cannula and distal end of the suction tubing will interconnect to one another via a ball and socket arrangement to thus enable such respective elements to rotate freely, preferably about a common axis, relative one another.

The liposuction cannula and suction tubing may be fabricated from known materials in the art, and may be formed from a sterilizable material to thus enable the same to be utilized repeatedly or fashioned to be disposable in nature after a single use.

### BRIEF DESCRIPTION OF THE DRAWINGS

These as well as other features of the present invention will become more apparent upon reference to the drawings wherein:

FIG. 1 is a perspective view of a liposuction cannula operatively interconnected with a section of suction tubing, the liposuction cannula and suction tubing being operatively interconnected via an interface constructed in accordance with a preferred embodiment of the present invention.

FIG. 2 is an enlarged cross-sectional view taken along line 2-2 of FIG. 1.

FIG. 3 is a cross-sectional view showing a coupling interposed between a liposuction cannula and a section of suction tubing, said coupling being constructed in accordance with a preferred embodiment of the present invention.

FIG. 4 is a cross-sectional view showing a ball and socket-type interface between a liposuction cannula that is constructed in accordance with another embodiment of the present invention.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

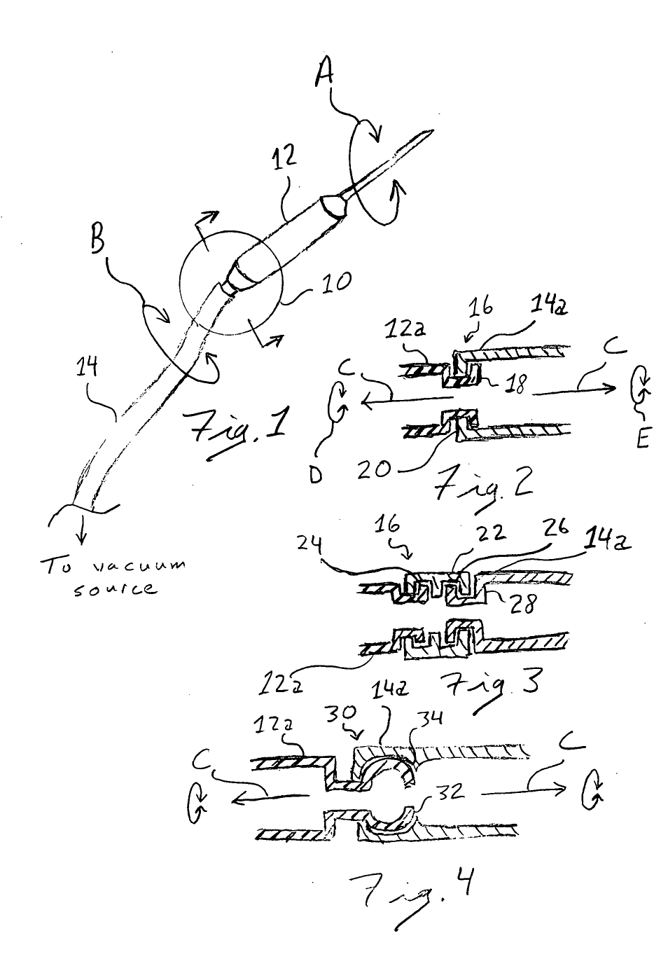

Referring now to the drawings, and initially to FIG. 1, there is perspectively illustrated a liposuction cannula 12 having a section of suction tubing 14 operatively connected thereto and extending therefrom. At the juncture between the liposuction cannula and suction tubing is an interface 10 that is operative to enable the liposuction and cannula and/or suction tubing to rotate freely relative one another, as reflected by circular paths A,B, about the juncture at which the suction tubing is coupled to the cannula. In this regard, by enabling such elements, namely, the liposuction cannula 12 and suction tubing 14, to rotate freely relative one another about the juncture at which the same are operatively coupled, any type of twisting or kinking of the suction tubing is substantially, if not completely, eliminated.

With respect to the interface 10 operative to provide such rotational movement at the juncture at which the liposuction cannula is coupled to the suction tubing, the same may comprise any of a variety of simple couplings or fittings known in the art. Illustrative of such embodiments include those depicted in FIGS. 2-4, respectively.

With respect to the former, such interface 10 may comprise a simple coupling 16 between the proximal end 12a of the liposuction cannula 12 with the distal-most end 14a of the suction tubing 14. In such embodiment, the liposuction cannula 12 is provided with an annular collar 18 about which is attached the distal-most end 14a of the suction tubing 14, the latter having an annular rim 20 formed about the distal end thereof to interconnect with the annular collar 18 formed upon the liposuction cannula. As will be appreciated by those skilled in the art, by virtue of such interconnection, the liposuction cannula 12 is freely able to rotate about axis “C” in the manner indicated by the letter “D” whereas the lumen of the suction tubing is also able to rotate about axis “C” in the manner illustrated by letter “E”.

In an alternative embodiment depicted in FIG. 3, the coupling 16 will comprise a fitting 22 formed as a separate component'and interconnectable intermediate the proximal end 12a of the liposuction cannula 12 and the distal end 14a of the suction tubing 14. In such embodiment, fitting 22 will be provided with a first annular groove 24 operative to engage with an annular collar 18 formed on the proximal end of the liposuction cannula 12 and enable the cannula to rotate in the manner described with respect to FIG. 2. Likewise, the fitting will interconnect with the distal-most end 14a of suction tubing 14 via a second annular groove 26, the latter engageable with annular collar 28, to enable the same also to rotate in the manner discussed above. To facilitate such rotational movement, it is expressly contemplated that the couplings or fittings of the present invention may be provided with ball bearings or other similar structures that can facilitate rotational movement of the liposuction cannula and/or suction tubing operatively interconnected therewith.

In yet a further embodiment depicted in FIG. 4, the interface comprises a ball and socket arrangement 30 whereby a ball type fitting 32 is formed upon the proximal end 12 of the liposuction cannula 12 about which is interconnected the distal-most end 14a of suction tubing 14. With respect to the latter, it is contemplated that the distal-most end 14a of suction tubing will be provided with a socket-like structure 34 operative to serve the dual purpose of forming a secure, fluid interconnection between such elements but at the same allowing the liposuction cannula and suction tubing to rotate relative one another about axis “C”.

The couplings or fittings defined in the interface 10 of the present invention may be fabricated from any of a variety of well-known materials. Specifically, it is contemplated that the couplings or fittings may be formed as integral components of either the liposuction cannula 12 and/or suction tubing 14, and thus either fabricated from a sterilizable material for reuse or otherwise fabricated for disposal following a single use. It will likewise be appreciated that the couplings or fittings of the present invention may be fabricated to conform to any of a variety of standardized dimensions to thus enable the same to be readily integrated with existing surgical equipment. brief description of the drawings

Additional modifications and improvements of the present invention may also be apparent to those of ordinary skill in the art. Thus, the particular combination of parts and steps described and illustrated herein is intended to represent only certain embodiments of the present invention, and is not intended to serve as limitations of alternative devices and methods within the spirit and scope of the invention. In this regard, it is expressly envisioned that the fittings, couplings, and/or interfaces of the present invention may take a variety of simple mechanical forms that will be readily appreciated by those skilled in the art. For example, it will be readily appreciated that the annular recess or groove in the annular collar formed upon the proximal end 12a of liposuction cannula 12 and suction tubing 14, as depicted in FIG. 2, can be reversed with the annular rim being formed on the proximal end 12a of liposuction cannula 12 and annular groove formed upon the distal end 14a of tubing 14. Likewise, the ball and socket arrangement 30 depicted in FIG. 4 may be reversed such that the socket structure is formed upon the proximal end 12a of liposuction cannula 12 whereas the ball type fitting is formed upon the distal end 14a of suction tubing 14.

1. An interface for operatively interconnecting a liposuction cannula to a free end of a section of suction tubing comprising:
a. an elongate section of suction tubing having a distal end and a lumen extending therethrough, said lumen defining an axis; and
b. a liposuction cannula having a coupling formed thereon and interconnectable with said distal end of said suction tubing; and
c. wherein said distal end of said suction tubing is interconnected to said coupling, said distal end of said suction tubing can freely rotate about said axis defined by said lumen.

2. The interface of claim 1 wherein said coupling is detachably fastenable to said liposuction cannula.

3. The interface of claim 1 wherein said coupling is sterilizable.

4. The interface of claim 1 wherein said coupling is integrally formed upon said liposuction cannula.

5. A liposuction cannula/suction tubing interface operative to provide rotational movement comprising:
a. a liposuction cannula having a proximal end defining an axis extending axially therethrough;
b. an elongate section of suction tubing having a lumen extending therethrough and a distal end defining an opening and axial passageway; and
c. a fitting formed upon said proximal end of said cannula for interconnecting with said distal end of said section of suction tubing and operative to align said axis extending from said proximal end of said liposuction cannula with said axial passageway defined by said distal end of said section of suction tubing, said fitting further being operative to permit the distal end of said section of suction tubing to rotate about said axis extending from said proximal end of said liposuction cannula about the juncture at which the tubing is coupled to said cannula relative said proximal end of said cannula.

6. The interface of claim 5 wherein said fitting is detachably fastenable to said liposuction cannula.

7. The interface of claim 5 wherein said mechanism is formed from a sterilizable material.

8. The interface of claim 5 wherein said fitting is formed as a ball member having an axial passageway extending therethrough and said distal end of said section of suction tubing defines a socket operatively interconnectable with such ball member.

9. A suction tube for use with a liposuction cannula comprising;
a. an elongate section of suction tubing defining a first end and a distal end and a lumen extending along the length thereof;
b. a fitting formed upon said distal end of said section of suction tubing operative to fluidly interconnect with a liposuction cannula and enable said liposuction cannula and section of suction tubing to independently rotate relative one another about a common axis.

10. The interface of claim 1 wherein said coupling formed upon said liposuction cannula comprises an annular groove; and said distal end of said section of suction tubing is provided with an annular collar operative to fit about and nest within said annular groove.

11. An interface for operatively interconnecting a liposuction cannula to a free end of a section of suction tubing comprising:
a. a liposuction cannula having a distal end for interconnecting with a section of suction tubing;
b. a section of suction tubing having a distal end and a lumen extending therethrough, said distal end having a coupling formed thereon for interconnecting with said proximal end of said liposuction cannula such that when said proximal end of said liposuction cannula is interconnected to said coupling, said proximal end of said liposuction cannula can freely rotate about said axis extending therethrough.

12. The interface of claim 1 wherein said coupling is integrally formed upon said distal end of said section of suction tubing.
